(19) United States
(12) Patent Application Publication
(10) Pub. No.: US 2012/0088969 A1
(43) Pub. Date: Apr. 12, 2012

(54) ENDOSCOPE APPARATUS

(76) Inventors: Masayuki TAKAHIRA, Ashigarakami-gun (JP); Masayuki Kawata, Ashigarakami-gun (JP); Issei Suzuki, Ashigarakami-gun (JP)

(21) Appl. No.: 13/270,742

(22) Filed: Oct. 11, 2011

(30) Foreign Application Priority Data
Oct. 12, 2010 (JP) 2010-230067

(51) Int. Cl. A61B 1/045

(52) U.S. Cl. 600/109

(57) ABSTRACT

An endoscope apparatus according to the subject matter includes: an illumination device configured to emit light to an object; an imaging device configured to generate a color imaging signal; a normal image generation device configured to generate a normal image from the color imaging signal; a spectral image generation device configured to generate a spectral image corresponding to a preset optical narrow band from the imaging signal; a display device configured to display a main screen region and a sub-screen region having a size less than a size of the main screen region; a switching signal generation device configured to generate a switching signal; and a display control device configured to control the display device so as to selectively switch between a region for displaying the normal image and a region for displaying the spectral image based on the switching signal.

### BACKGROUND OF THE INVENTION

1. Field of the Invention

The presently disclosed subject matter relates to an endoscope apparatus, and more particularly to an image processing technique and a screen display technique for use in an endoscope apparatus for medical treatment and other purposes.

2. Description of the Related Art

The endoscope apparatus for medical treatment and other purposes includes an insertion portion inserted into an interior of body. The end of the insertion portion includes an illumination window and an imaging apparatus. Under illumination light emitted through the illumination window, the interior of the body is imaged by the imaging apparatus. An imaging signal obtained by the imaging apparatus undergoes a predetermined image processing to be displayed on a monitor as an imaged image. Thus, an operator can observe the interior of the body by viewing the image displayed on the monitor.

The endoscope apparatus is used for observation of a wide variety of regions. For example, an upper endoscope is used for observation of plurality of regions such as a region from esophagus to stomach, a region from stomach to duodenum, a region from duodenum to stomach, and a region from stomach to esophagus. In addition, the upper endoscope is used for various observation purposes. For example, screening is performed to determine whether there is a problem region or not by a distant observation (a wide range of observation) or an entire observation. Then, the problem region is diagnosed by a near observation or a magnified observation.

Further, there has been proposed an apparatus which emits light of a specific frequency (wavelength) band to a region to be observed for imaging, generates a spectral image corresponding to the frequency band from an imaging signal, and displays the spectral image on a display apparatus. The observation using the spectral image enables observation of a blood vessel which cannot be observed by a normal image.

Japanese Patent Application Laid-Open No. 2002-95635 discloses an endoscope apparatus configured to be able to separately observe a body cavity tissue at a layer level using filters with different spectral properties to emit narrow-band light with a limited pass band.

Japanese Patent Application Laid-Open No. 2003-93336 discloses an electronic endoscope apparatus configured to emit light (white light) from an illumination light source to the interior of a body to be observed, obtain a color image signal by a solid-state imaging device, generate a spectral image from the color image signal, and display by switching the normal image and the spectral image on a display monitor. The electronic endoscope apparatus disclosed in Japanese Patent Application Laid-Open No. 2003-93336 is configured to estimate a signal reflected from a living body by matrix processing when light having a narrow-band wavelength is emitted.

Japanese Patent Application Laid-Open No. 2006-239206 discloses an endoscope apparatus having a color space conversion processing circuit forming a spectral image from matrix coefficients corresponding to RGB signals and λ₁, λ₂, and λ₃ signals in the selected three wavelength ranges. The endoscope apparatus disclosed in Japanese Patent Application Laid-Open No. 2006-239206 is configured such that when a spectral image is displayed on a display apparatus, a plurality of wavelength sets (λ₁, λ₂, and λ₃) of spectral wavelengths allocated to (Rₛ, Gₛ, and Bₛ) of the display apparatus is provided in advance; a plurality of spectral images corresponding to the plurality of wavelength sets is generated; the wavelength sets can be switched when an operator operates a selection switch; and the spectral image corresponding to the selected wavelength set can be cyclically switched.

### SUMMARY OF THE INVENTION

The aforementioned endoscope apparatuses having a display apparatus capable of displaying a large amount of information on one screen are required to satisfy the needs for the operator to perform a wide variety of observations to suit his or her desire or purpose without doing frequent switching operations or cumbersome operations.

Unfortunately, the endoscope apparatus disclosed in Japanese Patent Application Laid-Open No. 2002-95635 is required to have a filter for use in observation by narrow-band light and is required to mechanically switch the filter to change the band. Further, the endoscope apparatus cannot support simultaneous performance of a normal observation and a narrow-band observation or simultaneous recordings of an image obtained by the normal observation and an image obtained by the narrow band-observation. Furthermore, the endoscope apparatus is required to have a large number of band-pass filters to obtain a narrow-band wavelength.

Japanese Patent Application Laid-Open No. 2003-93336 describes that a normal image and a spectral image can be alternately displayed or simultaneously displayed, but does not disclose a specific method of switching the display or a specific method of simultaneous display.

The endoscope apparatus disclosed in Japanese Patent Application Laid-Open No. 2006-239206 is configured to be able to display only one spectral wavelength set, and thus has a difficulty in satisfying the needs for a wide variety of observations such as for simultaneously displaying a normal image and a spectral image and simultaneously performing a normal observation and a narrow band-observation.

In view of such circumstances, it is an object of the presently disclosed subject matter to provide an endoscope apparatus satisfying the needs for a wide variety of observations without frequent switching operations or cumbersome operations.

In order to achieve the above object, an endoscope apparatus according to the presently disclosed subject matter includes: an illumination device emitting light to an object; an imaging device generating a color imaging signal by imaging the light reflected from the object irradiated by light emitted from the illumination device; a normal image generation device generating a normal image by performing normal image processing on the imaging signal obtained by the imaging device; a spectral image generation device generating a spectral image corresponding to a preset optical narrow band by subjecting the imaging signal obtained by the imaging device to matrix processing using matrix data of the optical narrow band; a display device displaying a main screen region and a sub-screen region having a size less than the size of the main screen region; a switching signal generation device generating a switching signal for switching a display content of the main screen region and the sub-screen region; and a display control device controlling a screen display of the display device such that the normal image is displayed in any one of the main screen region and the sub-screen region, while the spectral image is displayed in the other region; and based on the switching signal generated by the switching signal generation device, control is performed so as to selectively switch between a region for displaying the normal image and a region for displaying the spectral image.

The presently disclosed subject matter provides a display device having a main screen region and a sub-screen region having a size less than the size of the main screen region, wherein either one of the normal image and the spectral image is displayed in one region and an image different from the image displayed in the one region is displayed in the other region, thus allowing the operator's attention to be concentrated on one region during simultaneous observations of the normal image and the spectral image.

In a state in which a normal image is displayed in the main screen region and a spectral image is displayed in the sub-screen region, the operator can view the display of the sub-screen region to determine whether a close investigation is required or not. If required, the operator can switch the display of the main screen region from the normal image to the spectral image. Further, in a state in which a spectral image is displayed in the main screen region and a normal image is displayed in the sub-screen region, the operator can view the normal image during observation of the spectral image, thereby securing safety during observation of the spectral image having less information than the normal image.

### BRIEF DESCRIPTION OF THE DRAWINGS

FIG. 1 is an entire configuration view of an endoscope according to embodiments of the presently disclosed subject matter;

FIG. 2 is a plan view illustrating a configuration of a distal end face illustrated in FIG. 1;

FIG. 3 is a sectional view illustrating an internal structure of a distal end hard portion illustrated in FIG. 1;

FIG. 4 is a block diagram illustrating a schematic configuration of an imaging unit and an image processing system of the endoscope apparatus illustrated in FIG. 1;

FIG. 5 is an explanatory drawing illustrating a screen layout of an observation monitor according to a first embodiment;

FIG. 6 is a flowchart illustrating a flow of a switching control of a screen display illustrated in FIG. 5;

FIG. 7 is an explanatory drawing illustrating a screen layout of an observation monitor according to a second embodiment;

FIG. 8 is a flowchart illustrating a flow of a switching control of the screen display illustrated in FIG. 7;

FIG. 9 is an explanatory drawing illustrating a screen layout of an observation monitor according to a third embodiment;

FIG. 10 is a flowchart illustrating a flow of a switching control of the screen display illustrated in FIG. 9;

FIG. 11 is a flowchart illustrating a flow of a switching control of the screen display during freeze operation;

FIG. 12 is a flowchart illustrating another flow of the switching control of the screen display during freeze operation;

FIG. 13 is an explanatory drawing of a screen layout according to a variation of the presently disclosed subject matter;

FIG. 14 is an explanatory drawing of a screen layout according to another variation of the presently disclosed subject matter;

FIG. 15 is an explanatory drawing of an isotropic mask for use in the screen display; and

FIG. 16 is an explanatory drawing of an anisotropic mask for use in the screen display.

### DETAILED DESCRIPTION OF THE PREFERRED EMBODIMENTS

Now, preferred embodiments of the presently disclosed subject matter will be described in detail referring to the accompanying drawings.

(Entire Configuration of Endoscope Apparatus)

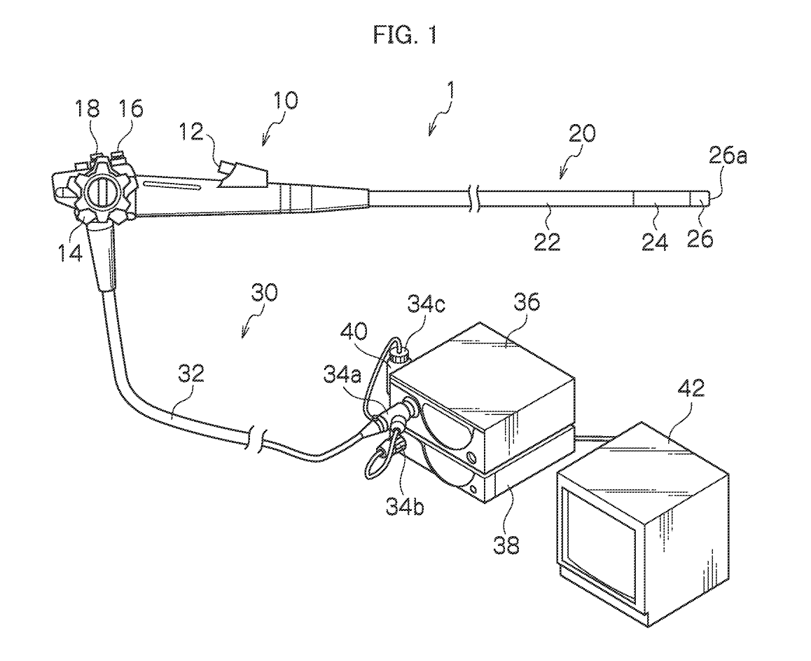

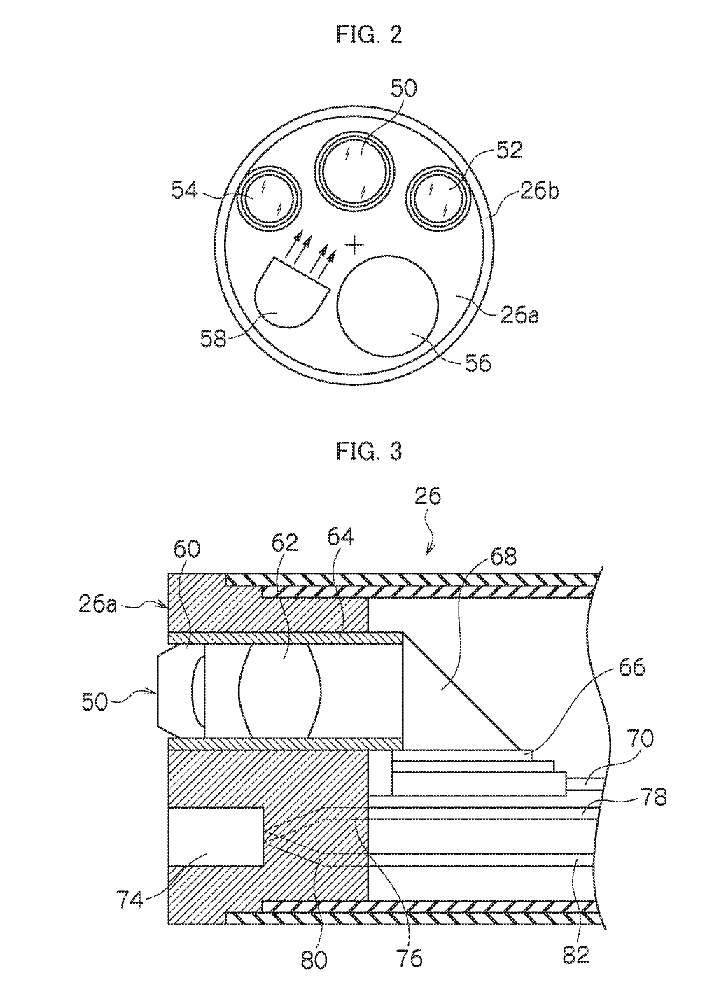

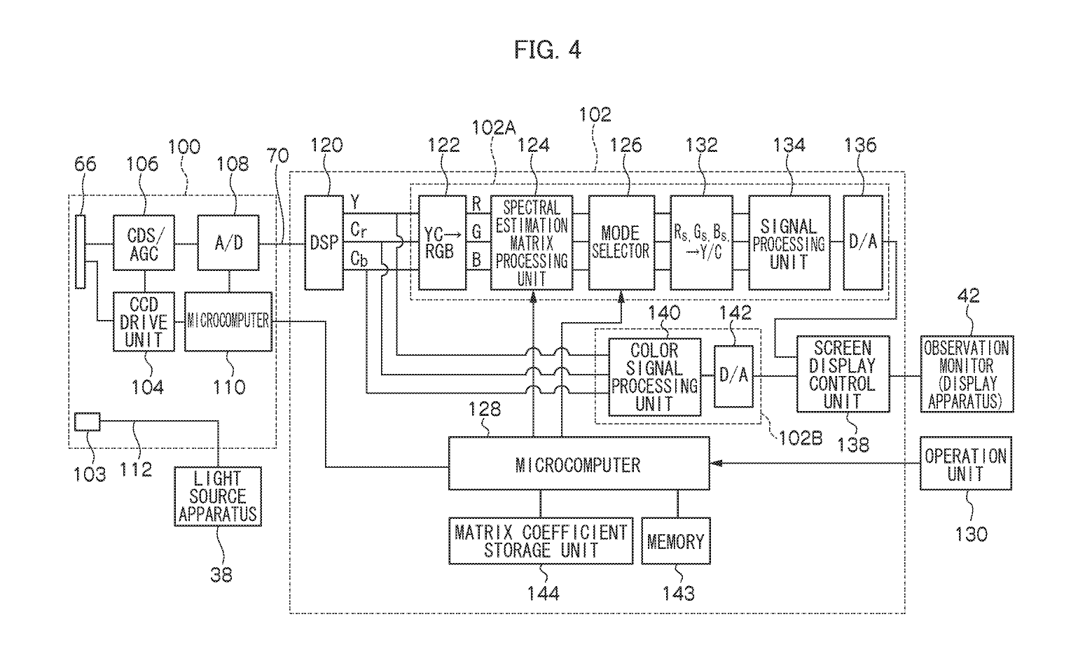

FIG. 1 is an entire configuration view illustrating an endoscope apparatus according to embodiments of the presently disclosed subject matter. An endoscope apparatus 1 illustrated in FIG. 1 includes an electronic endoscope for extracting an object image of an interior of a body cavity as an electronic image. The electronic endoscope includes: an operation portion 10 for an operator to perform a desired operation; an insertion portion 20 to be inserted into the interior of the body cavity; and a connection portion 30 for connecting the electronic endoscope to a peripheral apparatus. The peripheral apparatus includes: a processor apparatus 36 including an image processing unit and a control unit; a light source apparatus 38 connected to an illumination optical system (not illustrated in FIG. 1, but illustrated by reference numeral 103 in FIG. 4) disposed on a rear side of an illumination window (not illustrated in FIG. 1, but illustrated by reference numerals 52 and 54 in FIG. 2); an observation monitor 42 for displaying an image of the interior of the body cavity; and an air/liquid feed unit 40 supplying a cleaning fluid and air to be sprayed on an observation window (not illustrated in FIG. 1, but illustrated by reference numeral 50 in FIG. 2).

The operation portion 10 of the electronic endoscope includes: a forceps inlet 12 for inserting a treatment instrument; an angle knob 14 operated to bend a distal end of the insertion portion 20 upward, downward, left, and right; an air/liquid feed button 16 operated to spray water or air from a nozzle (not illustrated in FIG. 1, but illustrated by reference numeral 58 in FIG. 2) disposed in the distal end of the insertion portion 20 to clean an observation window disposed in the distal end of the insertion portion 20; and a suction button 18 for suction through a forceps outlet (not illustrated in FIG. 1, but illustrated by reference numeral 56 in FIG. 2) disposed in the distal end of the insertion portion 20.

The insertion portion 20 of the electronic endoscope has a predetermined diameter, has a substantially circular tube-like cross-section, and is integrally connected to a distal end of the operation portion 10. The insertion portion 20 includes: a flexible portion 22 having flexibility; a curvable curved portion 24 disposed at a distal end of the flexible portion 22; and a distal end hard portion 26 disposed at a distal end of the curved portion 24.

The flexible portion 22 of the electronic endoscope is made of a flexible tube and is integrally connected to the distal end of the operation portion 10. The flexible portion 22 occupies most of the insertion portion 20. The curved portion 24 is curvably configured and is integrally connected to the distal end of the flexible portion 22. The curved portion 24 is curved upward, downward, left, and right in conjunction with the operation of the angle knob 14 disposed in the operation portion 10. Thus, the distal end hard portion 26 can be oriented in a desired direction inside the body cavity by curving the curved portion 24 in a desired direction. The distal end hard portion 26 is made of a hard material such as a metal (e.g., stainless steel) into a cylindrical shape, and is integrally connected to the distal end of the curved portion 24.

The connection portion 30 of the electronic endoscope includes: a universal cord 32 continuously connected to the operation portion 10; and a plurality of connectors disposed in a distal end of the universal cord 32. The connector includes: a processor connector 34a for connecting to the processor apparatus 36; a light source connector 34b for connecting to the light source apparatus 38; and an air/liquid feed connector 34c for connecting to the air/liquid feed unit 40 disposed in a casing housing the processor apparatus 36 therein.

The observation monitor (display apparatus) 42 is a display device for displaying an object image of the interior of the body cavity. The operator performs screening while observing the image displayed on the observation monitor 42.

FIG. 2 is a plan view illustrating a structure of a distal end face 26a of the distal end hard portion 26 illustrated in FIG. 1. The distal end face 26a illustrated in FIG. 2 has a substantially circular planar shape and is disposed in a position closer to the outer periphery of the distal end face 26a. The distal end face 26a includes: an observation window 50 for observing a region to be observed; a pair of illumination windows 52 and 54 sandwiching the observation window 50 therebetween, disposed in a position closer to the outer periphery, and emitting illumination light to the region to be observed; a forceps outlet 56 as an outlet of a treatment instrument inserted from the forceps inlet 12 (see FIG. 1); and a nozzle 58 for spraying cleaning fluid and air on the observation window 50.

The nozzle 58 is arranged such that a spout (unillustrated) thereof is oriented toward the observation window 50. The forceps outlet 56 is arranged adjacent to the nozzle 58. An edge portion 26b in the outer periphery of the distal end face 26a is subjected to R-chamfering with a predetermined diameter.

The illumination optical system is arranged on a rear side of (inside) each of the pair of the illumination windows 52 and 54 illustrated in FIG. 2. The illumination optical system is connected to a light guide (not illustrated in FIG. 2, but illustrated by reference numeral 112 in FIG. 4) disposed inside the insertion portion 20 illustrated in FIG. 1. When the light source connector 34b of the connection portion 30 is connected to the light source apparatus 38, the illumination optical system is connected to a light-source lamp (unillustrated) housed in the light source apparatus 38. Consequently, when the light-source lamp of the light source apparatus 38 is turned on, the light from the light-source lamp is guided to the illumination optical system through the light guide. Then, the light guided to the illumination optical system is emitted toward the region to be observed through the illumination windows 52 and 54 illustrated in FIG. 2. Examples of the light source type applied to the present embodiment include white light (including incomplete white light).

The forceps outlet 56 illustrated in FIG. 2 is connected to the forceps inlet 12 of the operation portion 10 through a forceps channel (unillustrated) disposed inside the insertion portion 20 illustrated in FIG. 1. The treatment instrument such as a forceps inserted from the forceps inlet 12 protrudes from the forceps outlet 56 illustrated in FIG. 2.

The nozzle 58 is arranged so as to protrude from the distal end face 26a of the distal end hard portion 26 and has a spout (unillustrated) oriented toward the observation window 50. The spout is connected to the air/liquid feed unit 40 (see FIG. 1) through an air feed passage and a feed water flow inside the distal end hard portion 26, the flexible portion 22, and the connection portion 30. When the air/liquid feed button 16 disposed in the operation portion 10 is operated, air or water (cleaning fluid) supplied from the air/liquid feed unit 40 is selectively fed and the air or the water is sprayed from the spout of the nozzle 58 toward the observation window 50.

FIG. 3 is a sectional view illustrating an internal structure of the distal end hard portion 26 (a sectional view along the sectional line connecting the center of the observation window 50 and the center of the nozzle 58 illustrated in FIG. 2). As illustrated in FIG. 3, the observation window 50 is integrally configured with a cover glass 60. The cover glass 60 includes thereinside an object optical system 64 including an object lens 62. The cover glass 60 may be a lens constituting part of the object optical system 64. In general, a plano-concave lens is used as the cover glass 60.

A solid-state imaging device (CCD (charge-coupled device)) 66 having an RGB (primary color type; red, green and blue) or CMY (complementary color type; cyan, magenta and yellow) color filter on an imaging surface thereof is disposed at a focusing position of the object optical system 64. Light incident on the object optical system 64 passes through the illumination windows 52 and 54 (see FIG. 2) and reaches a region to be observed. Then, the light reflected from the region to be observed is inflected by about 90° by a prism 68 and is incident on a light receiving surface of the solid-state imaging device 66. Thus, the solid-state imaging device 66 focuses an optical image of the region to be observed on the light receiving surface. The optical image of the observed region imaged on the light receiving surface of the solid-state imaging device 66 is converted to an electrical signal (imaging signal) by the solid-state imaging device 66 and is sent to the processor apparatus 36 through a signal line 70. The electrical signal is converted to a video signal by the processor apparatus 36 and is displayed on the observation monitor 42 as an observation image of the interior of the body cavity.

A joining tube 74 communicatively connected to the spout of the nozzle 58 (see FIG. 2) is formed below the object optical system 64 illustrated in FIG. 3. The joining tube 74 is connected to an air feed tube 78 through an air feed passage 76 and connected to a liquid feed tube 82 through a liquid feed passage 80 as illustrated by dotted lines in FIG. 3. The air feed tube 78 and the liquid feed tube 82 are connected to the air/liquid feed connector 34c through inside the flexible portion 22 (see FIG. 1).

(Description of Imaging Unit and Image Processing Unit)

Now, an imaging unit and an image processing unit of the endoscope apparatus according to the present embodiment will be described in detail. FIG. 4 is a block diagram illustrating a schematic configuration of an imaging unit 100 and an image processing unit 102. The imaging unit 100 is disposed in the distal end hard portion 26 illustrated in FIG. 1. The image processing unit 102 is disposed in the processor apparatus 36 illustrated in FIG. 1.

As illustrated in FIG. 4, the imaging unit 100 includes a solid-state imaging device 66, an illumination optical system 103, a CCD (charge-coupled device) drive unit 104, a CDS/AGC (correlated double sampling/automatic gain control) unit 106, an A/D conversion unit 108, and a microcomputer 110.

The illumination optical system 103 is connected to a light source in the light source apparatus 38 through the light guide 112. The solid-state imaging device 66 is driven by the CCD drive unit 104 in response to a drive pulse generated based on a predetermined synchronization signal (clock) to output an imaging signal by converting the optical image of the observed region to an electrical signal.

The imaging signal obtained by the solid-state imaging device 66 is sampled at a predetermined sampling period by a correlated double sampling processing unit of the CDS/AGC unit 106 and is subjected to amplification processing by an automatic gain control processing unit thereof. Further, an analog imaging signal is converted to a digital imaging signal by an A/D conversion unit 108. Thus configured imaging unit 100 is integratedly controlled by a microcomputer 110. The microcomputer 110 disposed in the imaging unit 100 as well as the image processing unit 102 is integratedly controlled by a microcomputer 128 integratedly controlling the image processing unit 102.

The image processing unit 102 is a signal processing unit performing a predetermined image processing on an imaging signal received from the imaging unit 100 through a signal line 70. As illustrated in FIG. 4, the image processing unit 102 includes a DSP (digital signal processor) 120 performing various image processing on a digitally-converted image signal. The DSP 120 forms and outputs a Y/C signal including a luminance (Y) signal and a color-difference ((Cᵣ(R−Y), C_{b}(B−Y)) signal from a signal output from the solid-state imaging device 66.

The image processing unit 102 according to the present embodiment includes a normal image generation unit 102B generating a normal image (moving picture) and a spectral image generation unit 102A generating a spectral image (spectral moving picture). The image processing unit 102 is configured so as to simultaneously display the normal image and the spectral image on the observation monitor 42. The following description will focus on an embodiment of generating and displaying a normal image (normal moving picture) and a spectral moving picture, but apparently a normal still image (normal still picture) may be generated and displayed instead of the normal moving picture, and a spectral still picture may be generated and displayed instead. Note that the term “normal image” in the present description is a concept including a normal moving picture and a normal still picture, and the term “spectral image” is a concept including a spectral moving picture and a spectral still picture.

The Y signal, Cᵣ signal, and C_{b} signal output from the DSP 120 are converted to RGB (or CMYK) signals by a first color conversion unit 122. Note that the DSP 120 is disposed in the image processing unit 102, but may be disposed in the imaging unit 100.

As illustrated in FIG. 4, the image processing unit 102 includes a spectral estimation matrix processing unit 124 disposed at a later stage of the first color conversion unit 122 outputting the RGB signals. The spectral estimation matrix processing unit 124 is a signal processing unit which performs processing for estimating a reflected signal under irradiation of a narrow-band light having the selected wavelength of λ₁, λ₂, and λ₃, and outputs a spectral image signal (signal estimated as the reflected signal under irradiation of the narrow-band light having the selected wavelength of λ₁, λ₂, and λ₃) corresponding to the selected wavelength of λ₁, λ₂, and λ₃. The “spectral estimation matrix processing” according to the present embodiment refers to processing for estimating a reflected signal under irradiation of a narrow-band light (corresponding to an optical narrow-band) from a color imaging signal (imaging signal corresponding to an optical wide band) obtained under irradiation of white light using known information such as a spectral reflectance of a body (living body) to be observed, spectral properties of an illumination (light source apparatus 38), and spectral properties of a color filter installed in the color imaging device.

A mode selector 126 disposed at a later stage of the spectral estimation matrix processing unit 124 selects either one of a spectral image (monochrome mode) of a certain wavelength range (narrow band) and a spectral image (three-color mode) including three wavelength ranges based on a control signal received from the microcomputer 128. More specifically, when an operator selects the monochrome mode using the operation portion 130, the selection information is sent to the mode selector 126 through the microcomputer 128. Then, the spectral estimation matrix processing unit 124 generates the spectral image signal (any one of λ₁, λ₂, and λ₃) of the monochrome mode. When the operator selects the three-color mode using the operation portion 130, the selection information is sent to the mode selector 126 through the microcomputer 128. Then, the spectral estimation matrix processing unit 124 generates the spectral image signal (λ₁, λ₂, and λ₃) of the three-color mode.

The spectral image signal output from the spectral estimation matrix processing unit 124 enters a second color conversion circuit 132, in which the image signal (λ₁, λ₂, and λ₃) of one wavelength range or three wavelength ranges is converted to a signal of Rₛ(=λ₁), Gₛ(=λ₂), and Bₛ(=λ₃) for processing corresponding to a conventional RGB signal. Further, a signal processing unit 134 converts the Rₛ, Gₛ, Bₛ signal to a Y/C signal and performs other various signal processing (mirror image processing, mask generation, character generation, and the like). The spectral image signal output from the signal processing unit 134 enters a D/A conversion unit 136, in which the image signal is converted to an analog signal and sent to a screen display control unit 138. The first color conversion unit 122, the spectral estimation matrix processing unit 124, the mode selector 126, the second color conversion circuit 132, the signal processing unit 134, and the D/A conversion unit 136 function as the spectral image generation unit 102A.

Meanwhile, the Y signal, Cᵣ signal, and C_{b} signal output from the DSP 120 is sent to a color signal processing unit 140. The color signal processing unit 140 generates a normal color moving picture (normal moving picture) signal based on the Y signal, Cᵣ signal, and C_{b} signal output from the DSP 120. Then, the D/A conversion unit 142 converts the normal moving picture signal from digital to analog. Note that in an embodiment having a digital observation monitor 42, the D/A conversion units 136 and 142 are omitted. The color signal processing unit 140 and the D/A conversion unit 142 function as the normal image generation unit 102B.

The screen display control unit 138 controls the display format of the normal moving picture and the spectral moving picture based on a control signal received from the microcomputer 128. More specifically, when the operator operates the operation portion 130 for switching operation of the screen display format of the observation monitor 42, a control signal corresponding to the switching operation is sent from the microcomputer 128 to the screen display control unit 138. In response to the control signal, the screen display control unit 138 switches the screen display format of the observation monitor 42. Note that the detail about the switching control of the screen display format of the observation monitor 42 will be described later.

The operation portion 130 is used for switching the aforementioned spectral image, switching between a split screen display and a batch screen display described later, and selection of screen. The operation portion 130 may include at least one of a scope switch such as a button switch and a toggle switch, a pointing device such as a joystick and a mouse, and a touch panel. When the operation portion 130 is operated, an operation signal is sent to the microcomputer 128. In response to the operation signal, the microcomputer 128 sends a command signal to each unit corresponding to the operation signal.

Further, the operation portion 130 includes a freeze operation button for use in freeze operation described later. When the freeze operation button is operated, a normal still image (normal still picture) and a still image of the spectral image (spectral still picture) are stored in a predetermined memory at a timing when the freeze operation is effective. Either one of the normal still picture and the spectral still picture is displayed on the observation monitor 42 according to the screen display control during freeze operation.

The memory 143 includes a temporary storage memory as a work region for signal processing (image processing), a memory for storing various control parameters, and other memories. Writing to the memory 143 and reading from the memory 143 are controlled by the microcomputer 128. The memory 143 may be a semiconductor memory device such as a ROM (read-only memory) and a RAM (random access memory), or a magnetic memory device such as a hard disk drive.

FIG. 4 illustrates an embodiment in which the imaging unit 100 and the image processing unit 102 include the microcomputers 110 and 128 respectively, but the imaging unit 100 and the image processing unit 102 may be integratedly controlled by one microcomputer instead of the microcomputers 110 and 128. In such an embodiment, a microcomputer integratedly controlling the imaging unit 100 and the image processing unit 102 may be disposed in the imaging unit 100 (distal end hard portion 26) or in the image processing unit 102 (processor apparatus 36). Further, an imaging signal output from the A/D conversion unit 108 may be subjected to synchronization processing (color interpolating process) to acquire an RGB imaging signal. Furthermore, instead of the RGB (CMY) color filter, a predetermined spectral filter may be used.

(Description of Matrix Processing (Spectral Estimation Matrix Processing))

Now, the matrix processing (spectral estimation matrix processing, hereinafter may be referred to as simply “matrix processing”) according to the present embodiment will be described. The following description will focus on generation of a spectral image in a three-color mode. As illustrated in FIG. 4, the image processing unit 102 includes a matrix coefficient storage unit 144 in which matrix coefficient data for use in matrix processing for generating a spectral image signal is stored in a table format. The matrix coefficient storage unit 144 preliminarily stores matrix coefficients for a plurality of wavelength sets. An example of data table of the matrix coefficients is illustrated in the following Table 1.

TABLE 1

| PARAMETER | kₚᵣ | k_{pg} | k_{pb} |
|---|---|---|---|
| p1 | 0.000083 | −0.00188 | 0.003592 |
| . | . | . | . |
| . | . | . | . |
| . | . | . | . |
| p18 | −0.00115 | 0.000569 | 0.003325 |
| p19 | −0.00118 | 0.001149 | 0.002771 |
| p20 | −0.00118 | 0.001731 | 0.0022 |
| p21 | −0.00119 | 0.002346 | 0.0016 |
| p22 | −0.00119 | 0.00298 | 0.000983 |
| p23 | −0.00119 | 0.003633 | 0.000352 |
| . | . | . | . |
| . | . | . | . |
| . | . | . | . |
| p43 | 0.003236 | 0.001377 | −0.00159 |
| p44 | 0.003656 | 0.000671 | −0.00126 |
| p45 | 0.004022 | 0.000068 | −0.00097 |
| p46 | 0.004342 | −0.00046 | −0.00073 |
| p47 | 0.00459 | −0.00088 | −0.00051 |
| p48 | 0.004779 | −0.00121 | −0.00034 |
| p49 | 0.004922 | −0.00148 | −0.00018 |
| p50 | 0.005048 | −0.00172 | −3.6E−05 |
| p51 | 0.005152 | −0.00192 | 0.000088 |
| p52 | 0.005215 | −0.00207 | 0.000217 |
| . | . | . | . |
| . | . | . | . |
| . | . | . | . |
| p61 | 0.00548 | −0.00229 | 0.00453 |

The matrix coefficient data illustrated in Table 1 includes 61 wavelength range parameters p1 to p61 obtained by dividing the wavelength range from 400 nm to 700 nm at an interval of 5 nm. The parameters p1 to p61 include coefficients kₚᵣ, k_{pg}, and k_{pb} (p corresponds to p1 to p61 in Table 1) for matrix processing. The spectral estimation matrix processing unit 124 illustrated in FIG. 4 uses the coefficients kₚᵣ, k_{pg}, and k_{pb} and the RGB signal output from the first color conversion unit 122 to perform matrix processing by the following Expression (1).

$$( \begin{matrix}{λ}_{1} \\ {λ}_{2} \\ {λ}_{3}\end{matrix} ) = ( \begin{matrix}{k}_{1 r} & {k}_{1 g} & {k}_{1 b} \\ {k}_{2 r} & {k}_{2 g} & {k}_{2 b} \\ {k}_{3 r} & {k}_{3 g} & {k}_{3 b}\end{matrix} ) \times ( \begin{matrix}R \\ G \\ B\end{matrix} ) \qquad [ Expression 1 ]$$

For example, when parameters p21 (central wavelength of 500 nm), p45 (central wavelength of 620 nm), and p51 (central wavelength of 650 nm) is selected in Table 1, Expression 1 may be such that (−0.00119, 0.002346, 0.0016) of p21 is assigned to the coefficient (k₁ᵣ, k_{1g}, and k_{1b}), (0.004022, 0.000068, −0.00097) of p45 is assigned to the coefficient (k₂ᵣ, k_{2g}, and k_{2b}), and (0.005152, −0.00192, 0.000088) of p51 is assigned to the coefficient (k₃ᵣ, k_{3g}, and k_{3b}).

Note that when the spectral image signal of the monochrome mode is generated, the spectral image signal is set to any one of the λ₁, λ₂, and λ₃ in the Expression 1. The value may be a central wavelength or a maximum wavelength or a minimum wavelength. The image processing unit 102 according to the present embodiment is configured to be switchable between the monochrome mode and the three-color mode. Apparently, the image processing unit 102 may be fixed to either one of the monochrome mode and the three-color mode. The following description of the screen display switching will focus on an embodiment of generating a spectral moving picture in the three-color mode.

(Description of Screen Display Control (First Embodiment))

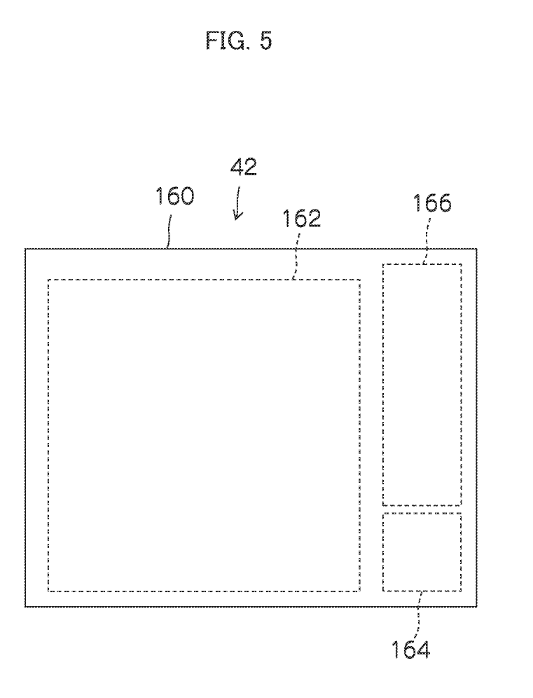

Now, the screen display control of the observation monitor 42 according to a first embodiment will be described in detail. FIG. 5 is an explanatory drawing illustrating a screen layout of the observation monitor 42. As illustrated in FIG. 5, a screen 160 displayed on the observation monitor 42 has a structure divided into a main screen region 162, a sub-screen region 164, and an information region 166. The screen 160 is configured to simultaneously display the main screen region 162, the sub-screen region 164, and the information region 166. The main screen region 162 is a region for displaying either one of the normal moving picture and the spectral moving picture, is arranged so as to include a central portion of the screen 160, and has about sixteen times the area of the sub-screen region 164. The size and the position of the main screen region 162 are fixed. A maximum resolution (e.g., 1600×1200 (UXGA (Ultra Extended Graphics Array))) of the screen 160 is applied to the main screen region 162.

The sub-screen region 164 is a region for displaying an image of the normal moving picture or the spectral moving picture, whichever is not displayed on the main screen region 162, is arranged in any one of the four corners of the screen 160, has about one-sixteenth of the area of the main screen region 162, and has the same angle of view (field of view) as that of the main screen region 162. The sub-screen region 164 may be configured such that the size and the position thereof are variable. For example, the sub-screen region 164 may be arranged in a central portion of the screen 160 in up and down directions of the figure. Further, the sub-screen region 164 may be configured to be moved in up and down directions of the screen 160 in the figure by operating an operation member such as a joystick. Furthermore, the sub-screen region 164 may have a lower resolution than that of the main screen region 162. Still further, the size ratio between the sub-screen region 164 and the main screen region 162 may be increased to a degree to allow the operator to recognize. When the size ratio between the sub-screen region 164 and the main screen region 162 is increased, the resolution of the sub-screen region 164 may be lowered than that of the main screen region 162.

An about 20-inch liquid crystal display (screen size example: 408 mm×306 mm) may be applied to the observation monitor 42.

The screen display format in the initial state of the observation monitor 42 according to the present embodiment is such that a normal moving picture is displayed on the main screen region 162 and a spectral moving picture is displayed on the sub-screen region 164. The information region 166 displays the wavelength information, the scope name, the imaging condition, the date, and other related information. Note that the format of the information region 166 appropriately changes according to the position and the size of the sub-screen region 164. For example, when the position of the sub-screen region 164 is changed, the information region 166 may be moved to a position not overlapped with the sub-screen region 164 or may be displayed to be overlaid on the main screen region 162 or the sub-screen region 164. Further, the information region 166 may be hidden.

The screen display control of the observation monitor 42 according to the present embodiment allows the operator to selectively switch between an image displayed on the main screen region 162 and an image displayed on the sub-screen region 164. For example, when the screen display is switched using the operation portion 130 illustrated in FIG. 4, a spectral moving picture (or a normal moving picture) displayed on the sub-screen region 164 is displayed on the main screen region 162, and a normal moving picture (or a spectral moving picture) displayed on the main screen region 162 is displayed on the sub-screen region 164.

When the screen display is switched again, the image displayed on the main screen region 162 is switched to the image displayed on the sub-screen region 164. Note that when the screen display is switched, the resolution of the image displayed on the main screen region 162 is changed to a maximum resolution. Note also that the resolution of the image changed from the main screen region 162 to the sub-screen region 164 may be lowered.

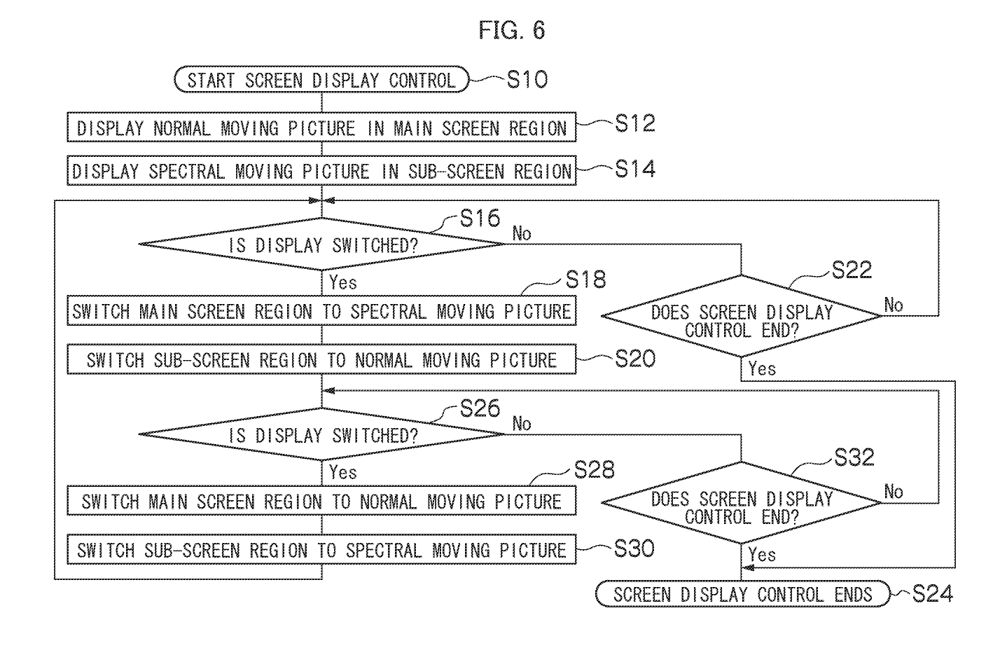

FIG. 6 is a flowchart illustrating a flow of the screen display control. When the screen display control starts (step S10), a normal moving picture is displayed on the main screen region 162 (step S12), and a spectral moving picture is displayed on the sub-screen region 164 (step S14). If the display is switched by the operator (Yes in step S16), the main screen region 162 is switched from the normal moving picture to the spectral moving picture (step S18), and the sub-screen region 164 is switched from the spectral moving picture to the normal moving picture (step S20). If the display is not switched (No in step S16), the process moves to step S22, in which a determination is made as to whether the screen display control ends or not. If a determination is made in step S22 that the screen display control ends (Yes in step S22), the screen display control ends (step S24). If a determination is made in step S22 that the screen display control does not end (No in step S22), the process returns to step S16, in which a determination is made as to whether the display is switched or not.

If the screen display is switched in steps S18 and S20, and then, the screen display is switched again (Yes in step S26), the main screen region 162 is switched from the spectral moving picture to the normal moving picture (step S28), and the sub-screen region 164 is switched from the normal moving picture to the spectral moving picture (step S30). Then, the process returns to step S16. If a determination is made in step S26 that the screen display is not switched (No in step S26), the process moves to step S32, in which a determination is made as to whether the screen display control ends or not. If a determination is made in step S32 that the screen display control ends (Yes in step S32), the screen display control ends (step S24). If a determination is made in step S32 that the screen display control does not end (No in step S32), the process returns to step S26.

Thus, the main screen region 162 and the sub-screen region 164 are configured to be visually different in size, allowing the operator to clearly differentiate the main screen region 162 from the sub-screen region 164 and thereby exerting an effect of concentrating the operator's attention on one screen. Thus, this configuration can satisfy the operator's request to focus on one screen during scope operation or screening observation.

In a case in which the normal moving picture is displayed on the main screen region 162 and the spectral moving picture is displayed on the sub-screen region 164, the operator can use the normal moving picture to perform screening by observing the normal moving picture. If a close investigation using the spectral moving picture is required, the operator can perform switching operation to display the spectral moving picture on the main screen region 162 to observe the spectral moving picture. On doing such a switching operation, the operator can quickly look at the spectral moving picture displayed on the sub-screen region 164 to determine whether the close investigation is required or not. Thus, this configuration can satisfy the operator's request to minimize the number of switching operations.

In a case in which the spectral moving picture is displayed on the main screen region 162 and the normal moving picture is displayed on the sub-screen region 164, the operator can perform screening by observing the spectral moving picture. Since the normal moving picture is displayed on the sub-screen region 164, safety can be secured. Specifically, the spectral moving picture has missing information, but simultaneously displaying the normal moving picture on the sub-screen region 164 can exert an effect of improving safety.

(Description of Screen Display Control (Second Embodiment))

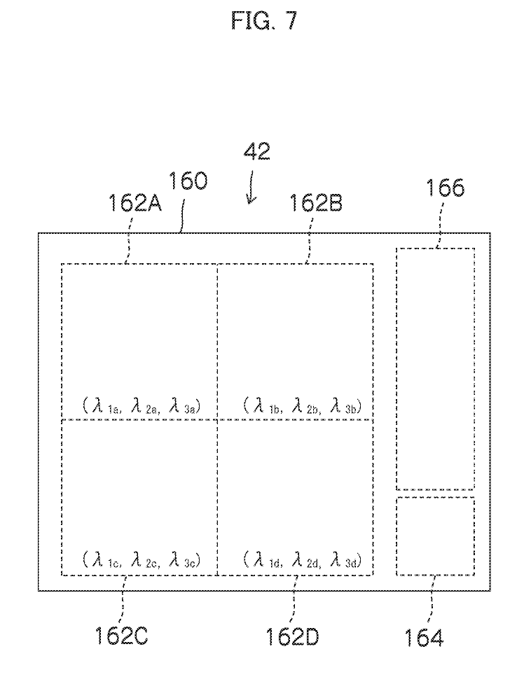

Now, the screen display control of the observation monitor 42 according to a second embodiment will be described. FIG. 7 is an explanatory drawing illustrating a screen layout of the observation monitor 42 according to the second embodiment. Note that in the following description, the same reference numerals or characters are assigned to the parts which are the same as or similar to those already described, and the description thereof is omitted.

The screen 160 illustrated in FIG. 7 is configured such that the main screen region 162 is divided into four regions: 162A, 162B, 162C, and 162D, each displaying a spectral moving picture having a different wavelength.

In a case in which a spectral moving picture is displayed in the main screen region 162, when the display mode is switched to a divided display mode, four kinds of spectral moving pictures corresponding to a plurality of preset wavelength sets (a, b, c, and d) are displayed in the respective divided regions 162A, 162B, 162C, and 162D. The spectral moving picture displayed in a first main screen region 162A corresponds to a wavelength (λ₁ₐ, λ₂ₐ, λ₃ₐ). The spectral moving picture displayed in a second main screen region 162B corresponds to a wavelength (λ_{1b}, λ_{2b}, λ_{3b}). The spectral moving picture displayed in a third main screen region 162C corresponds to a wavelength (λ_{1c}, λ_{2c}, and λ_{3c}). The spectral moving picture displayed in a fourth main screen region 162D corresponds to a wavelength (λ_{1d}, λ_{2d}, and λ_{6d}).

Specifically, four kinds of spectral images are generated according to the wavelength sets (a, b, c, and d) corresponding to the preset four wavelengths: a, b, c, and d. The matrix coefficient storage unit 144 illustrated in FIG. 4 stores matrix coefficients for generating spectral images corresponding to the plurality of wavelength sets (a, b, c, and d). The wavelength set is prepared for each region to be observed by an association such that a is standard, b designates a region for extracting a blood vessel, c designates a region for extracting a tissue, and d designates a region for extracting a difference between oxyhemoglobin and deoxyhemoglobin. Note that the association may further include a region for extracting a difference between a blood vessel and carotene; and a region for extracting a blood vessel between a cell.

For example, it is preferable that in a case in which the main screen region 162 is displayed as a single screen as illustrated in FIG. 5, a spectral image corresponding to the wavelength a (standard) is displayed; and when the main screen region 162 is switched to a divided display as illustrated in FIG. 7, four kinds of spectral images are displayed in the respective divided regions. Note that as such a display example, a thicker line is used for the outer frame, a character display is added, and other forms can be considered.

Note that the main screen region 162 may be divided into more number of regions to be associated with more number of wavelength sets including more number of wavelengths. The number of divisions of the main screen region 162 is preferably 4, 9, and 16 so as to maintain the aspect ratio (size ratio in vertical and horizontal directions) of each region. Note also that each divided region of the main screen region 162 is preferably larger in size than the sub-screen region 164. Thus, from the point of view of this, the number of divisions of the main screen region 162 is more preferably 4.

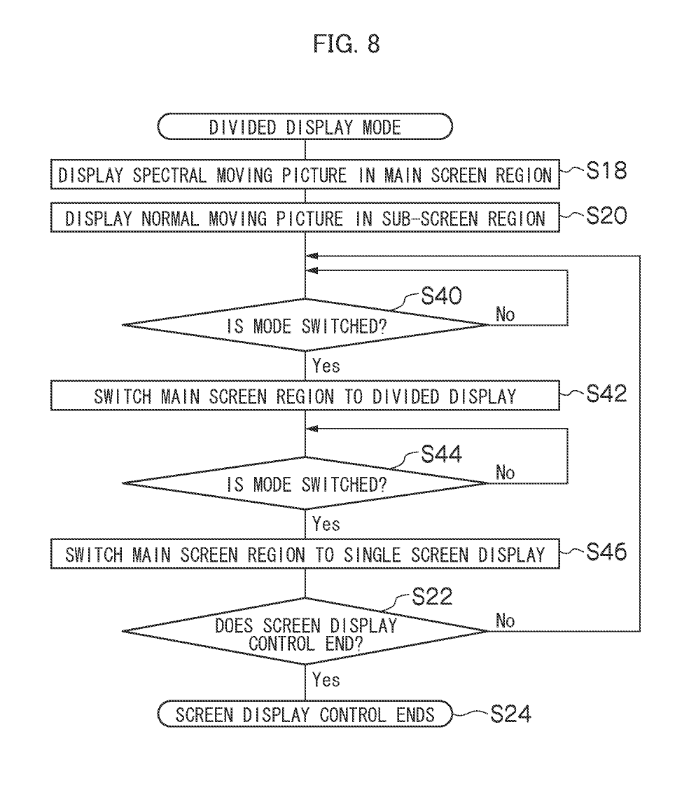

FIG. 8 is a flowchart illustrating a flow of the screen display control according to the second embodiment. Note that in FIG. 8, the same reference numerals or characters are assigned to the steps which are the same as or similar to those in FIG. 6, and the description thereof is omitted. In a case in which a spectral moving picture is displayed as a single screen display on the main screen region 162 and a normal moving picture is displayed in the sub-screen region 164 (steps S18 and 20), if the screen display mode is switched (Yes in step S40), the main screen region 162 is switched to a divided display (step S42). Then, the process moves to step S44. If the screen display mode is not switched (No in step S40), the process returns to step S40, in which monitoring is performed to determine whether the screen display mode is switched or not.

If a determination is made in step S44 that the screen display mode is switched (Yes in step S44), the main screen region 162 is switched to a single screen display (step S46). Then, the process moves to step S22. Note that when the main screen region 162 is switched from the divided display to the single screen display, the spectral moving picture corresponding to the wavelength before being switched to the divided display is displayed. Apparently, after the main screen region 162 is switched from the divided display to the single screen display, the spectral images corresponding to the wavelength sets (a, b, c, and d) may be sequentially switched and displayed.

In step S22, a determination is made as to whether the screen display control ends or not. If a determination is made in step S22 that the screen display control ends (Yes in step S22), the screen display control ends (step S24). If a determination is made in step S22 that the screen display control does not end (No in step S22), the process returns to step S40, in which the processes in steps S40 to S46 and S22 are repeated.

The screen display switching according to the second embodiment enables a simultaneous and parallel display of a plurality of spectral images (special images) to minimize a risk of missing observation of the operator and enables a simultaneous application of a plurality of diagnostic methods during screening.

(Description of Screen Display Control (Third Embodiment))

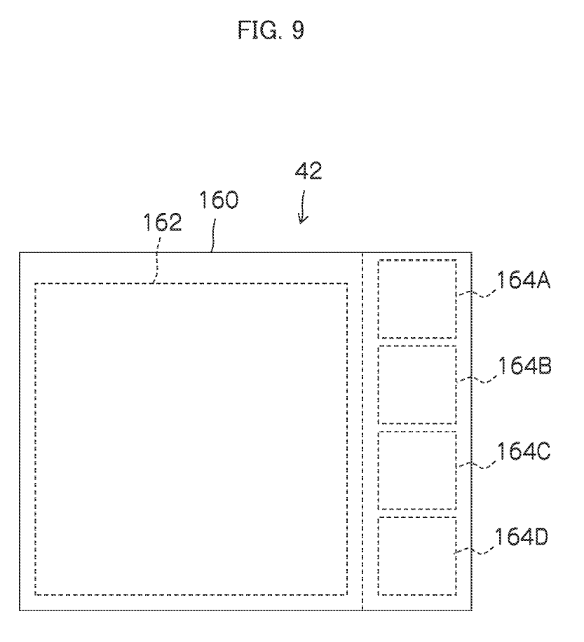

Now, the screen display control of the observation monitor 42 according to a third embodiment will be described. FIG. 9 is an explanatory drawing illustrating a screen layout of the observation monitor 42 according to the third embodiment. The screen 160 illustrated in FIG. 9 includes four sub-screen regions 164A, 164B, 164C, and 164D. The main screen region 162 displays one image selected from the images displayed in the four sub-screen regions 164A, 164B, 164C, and 164D. For example, a normal moving picture is displayed in a first sub-screen region 164A, and a spectral moving picture corresponding to one of the wavelength sets (a, b, and c) is displayed in a second sub-screen region 164B, a third sub-screen region 164C, and a fourth sub-screen region 164D respectively. When the first sub-screen region 164A is selected, the normal moving picture is always displayed in the main screen region 162. When any one of the second to fourth sub-screen regions 164B to 164D is selected, the selected spectral moving picture is displayed in the main screen region 162.

A touch panel display apparatus may be applied to the observation monitor 42 as a device for selecting one of the first to fourth sub-screen regions 164A to 164D. Specifically, when an operator touches a desired image in the first to fourth sub-screen regions 164A to 164D (selection screen), the touched image is displayed in the main screen region 162. Note that the image displayed in the main screen region 162 may be displayed in the sub-screen region 164 as is, or the image displayed in the main screen region 162 may be deleted from the sub-screen region 164.

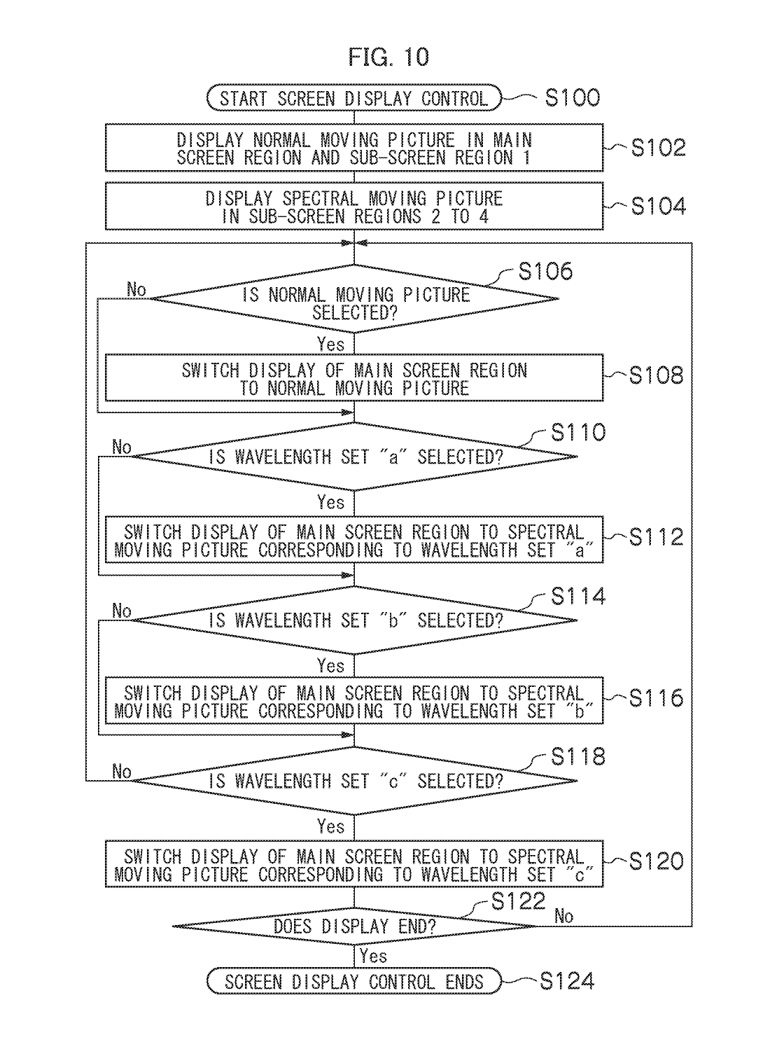

FIG. 10 is a flowchart illustrating a flow of the screen display switching control according to the third embodiment. When the screen display control starts (step S100), first, a normal moving picture and a plurality of spectral moving pictures are displayed on the selection screens (a first sub-screen region 164A to a fourth sub-screen region 164D) respectively. More specifically, the normal moving picture is displayed in the first sub-screen region 164A (step S102), three kinds of spectral moving pictures are displayed in the second to fourth sub-screen regions 164B to 164D (step S104) respectively. For example, spectral moving pictures corresponding to the wavelength sets (a, b, and c) is displayed in the second to fourth sub-screen regions 164B to 164D respectively.

Then, the process moves to step S106. If the normal moving picture is selected (Yes in step S106), the normal moving picture is displayed in the main screen region 162 (step S108), but the display in the first to fourth sub-screen regions 164A to 164D remains unchanged. If the normal moving picture is not selected (No in step S106), the process moves to step S110. If a spectral moving picture corresponding to wavelength a is selected (Yes in step S110), the spectral moving picture corresponding to wavelength a is displayed in the main screen region 162 (step S112). If a spectral moving picture corresponding to wavelength a is not selected (No in step S110), the process moves to step S114.

If a spectral moving picture corresponding to wavelength b is selected (Yes in step S114), the spectral moving picture corresponding to wavelength b is displayed in the main screen region 162 (step S116). If a spectral moving picture corresponding to wavelength b is not selected (No in step S114), the process moves to step S118. If a spectral moving picture corresponding to wavelength c is selected (Yes in step S118), the spectral moving picture corresponding to wavelength c is displayed in the main screen region 162 (step S120). If a spectral moving picture corresponding to wavelength c is not selected (No in step S118), the process returns to step S106.

In step S122, a determination is made as to whether the screen display control ends or not. If a determination is made that the screen display control ends (Yes in step S122), the screen display control ends (step S124). If a determination is made that the screen display control does not end (No in step S122), the process returns to step S106, in which the processes in steps S106 to S122 are repeated. Thus, one image is selected from the images displayed in the sub-screen regions 164A to 164D (selection screens), and the selected image is displayed in the main screen region 162.

Even if a certain image is displayed in the main screen region 162, the image can be switched to another image selected in the selection screen. Thus, the image displayed in the main screen region 162 is switched for each selection. Note that preferably the thickness and the color of the outer frame of the selected image are changed, and a character display is used to emphasize the selected image.

According to the screen display switching according to the third embodiment, candidate images to be displayed in the main screen can be displayed in selection screens, thus allowing the operator to instinctively select a desired image. Note that the screen display switching according to the third embodiment may be applied only for displaying the spectral image in the main screen region 162. In such an embodiment, for example, any one of the four spectral images corresponding to the respective wavelength sets (a, b, c, and d) is displayed in the main screen region 162, and the other three spectral images are displayed in the respective sub-screen regions 164B to 164D. When any one of the spectral images displayed in the sub-screen regions 164B to 164D is selected, the display screen is switched from the spectral image displayed in the main screen region 162 to the selected spectral image.

(Description of Screen Display Control During Freeze Operation)

Now, the screen display control during freeze operation will be described. The endoscope apparatus 1 according to the present embodiment is configured to display a still picture in the main screen region 162 when the freeze operation is performed to switch the screen display of the observation monitor 42 from moving picture display to still picture display.

For example, in a state in which a spectral moving picture is displayed in the main screen region 162 and a normal moving picture is displayed in the sub-screen region 164, when the freeze operation is performed on the screen 160 illustrated in FIG. 5, a still picture of the spectral image is displayed in the main screen region 162 and the display of the normal moving picture continues in the sub-screen region 164. In a state in which a normal moving picture is displayed in the main screen region 162 and a spectral moving picture is displayed in the sub-screen region 164, when the freeze operation is performed, a still picture of the normal image is displayed in the main screen region 162 and the display of the sub-screen region 164 is switched from the spectral moving picture to the normal moving picture.

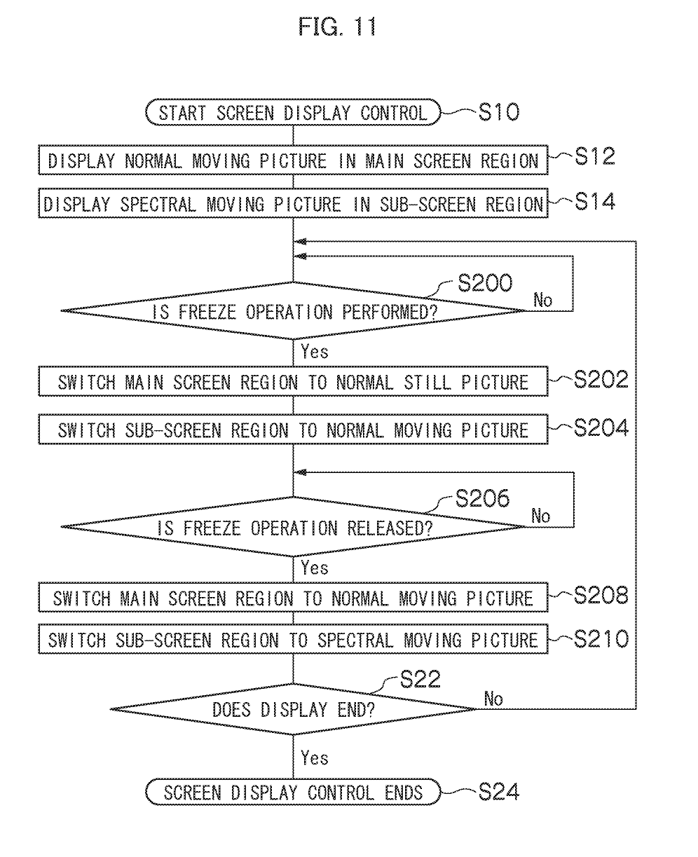

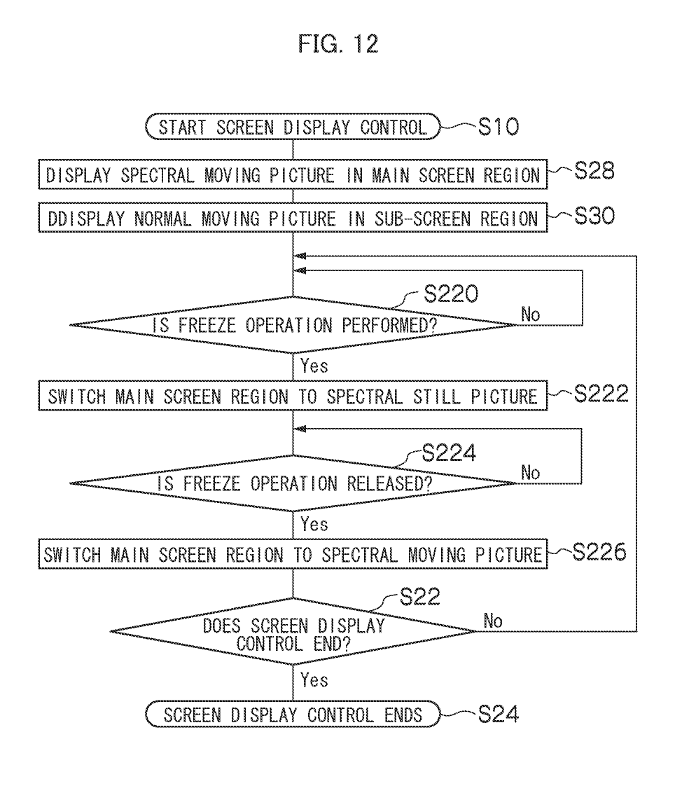

FIGS. 11 and 12 are a flowchart illustrating a flow of the screen display switching control during freeze operation. Note that in FIGS. 11 and 12, the same reference numerals are assigned to the steps which are the same as or similar to those in FIGS. 6 and 8.

As illustrated in FIG. 11, the screen display control starts (step S10). Then, a normal moving picture is displayed in the main screen region 162 (step S12) and a spectral moving picture is displayed in the sub-screen region 164 (step S14). Here, if the freeze operation is performed (Yes in step S200), the main screen region 162 is switched to the still picture of the normal image (still picture of the normal image at the timing when the freeze operation is effective) (step S202). Then, the sub-screen region 164 is switched from the spectral moving picture to the normal moving picture (step S204). Then, the process moves to step S206.

If the freeze operation is not performed (No in step S200), the process returns to step S200, in which monitoring continues until the freeze operation is performed. If the freeze operation is released (Yes in step S206), the main screen region 162 is switched from the normal still picture to the normal moving picture (step S208), and the sub-screen region 164 is switched from the normal moving picture to the original spectral moving picture (step S210). Then, the process moves to step S22.

If the freeze operation is not released (No in step S206), the process returns to step S206, in which monitoring continues until the freeze operation is released. If a determination is made in step S22 that the screen display control ends (Yes in step S22), the screen display control ends (step S24). If a determination is made in step S22 that the screen display control does not end (No in step S22), the process returns to step S200, in which the processes in steps S200 to S210 and S22 are repeated.

As illustrated in FIG. 12, the screen display control starts (step S10). Then, the main screen region 162 is switched to the spectral moving picture (step S28) and the sub-screen region 164 is switched to the normal moving picture (step S30). Here, if the freeze operation is performed (Yes in step S220), the main screen region 162 is switched to the spectral still picture (still picture of the spectral image at the timing when the freeze operation is effective) (step S222). The sub-screen region 164 continues to display the normal moving picture. Then, the process moves to step S224. If the freeze operation is not performed (No in step S220), the process returns to step S220, in which monitoring continues until the freeze operation is performed.

If the freeze operation is released (Yes in step S224), the main screen region 162 is switched to the spectral moving picture (step S226), and the sub-screen region 164 continues to display the normal moving picture. Then, the process moves to step S22. If the freeze operation is not released (No in step S224), the process returns to step S224, in which monitoring continues until the freeze operation is released. If a determination is made in step S22 that the screen display control ends (Yes in step S22), the screen display control ends (step S24). If a determination is made in step S22 that the screen display control does not end (No in step S22), the process returns to step S220, in which monitoring continues until the freeze operation is performed.

Thus, when the freeze operation is performed, the display of the main screen region 162 is switched from the normal moving picture to the normal still picture, and the spectral moving picture is switched to the spectral still picture, but the normal moving picture is always displayed in the sub-screen region 164, thereby securing the safety during freeze operation.

Further, the still picture observation and the moving picture observation can be easily alternately switched. Thus, the observation format can be appropriately changed according to the desire of the operator. Furthermore, the observation format can be changed by a button operation, and thus the observation format can be changed without cumbersome operations.

In the embodiment having a plurality of sub-screen regions 164A to 164D illustrated in FIG. 9, in a case in which a normal moving picture is displayed in the main screen region 162 and the sub-screen region 164A, and spectral moving pictures are displayed in the sub-screen regions 164B to 164D, when the freeze operation is performed, the display of the main screen region 162 is switched from the normal moving picture to the normal still picture and the sub-screen region 164A continues to display the normal moving picture.

Alternatively, in a state in which a normal moving picture is displayed in the main screen region 162 and the normal moving picture is not displayed in the sub-screen region 164 (sub-screen region 164 is turned off), when the freeze operation is performed, the display of the main screen region 162 is switched from the normal moving picture to the normal still picture and the normal image is displayed in the sub-screen region 164A.

Note that in response to the freeze operation, the display of the sub-screen regions 164B to 164D may be turned off and when the freeze operation is released, the display may return to the original screen display.

(Variation)

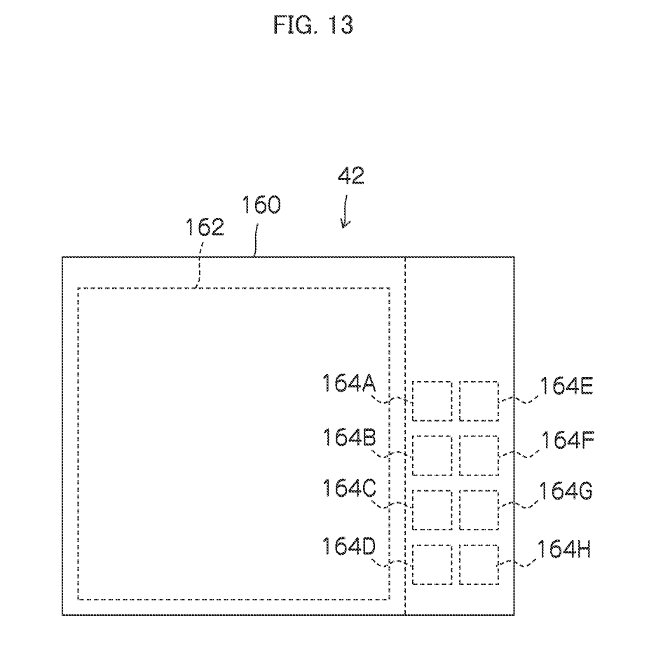

FIG. 13 is a variation of a screen layout according to the third embodiment. As illustrated in FIG. 13, an embodiment having a further more number of sub-screen regions may be preferred. In an example illustrated FIG. 13, the variation of the screen layout includes a first sub-screen region 164A displaying a normal moving picture and seven sub-screen regions (a second sub-screen region 164B to an eighth sub-screen region 164H) displaying seven spectral images. The variation may be configured such that when an image displayed in the main screen region 162 is selected, only one sub-screen region 164 as illustrated in FIG. 5 is displayed instead of the seven spectral images.

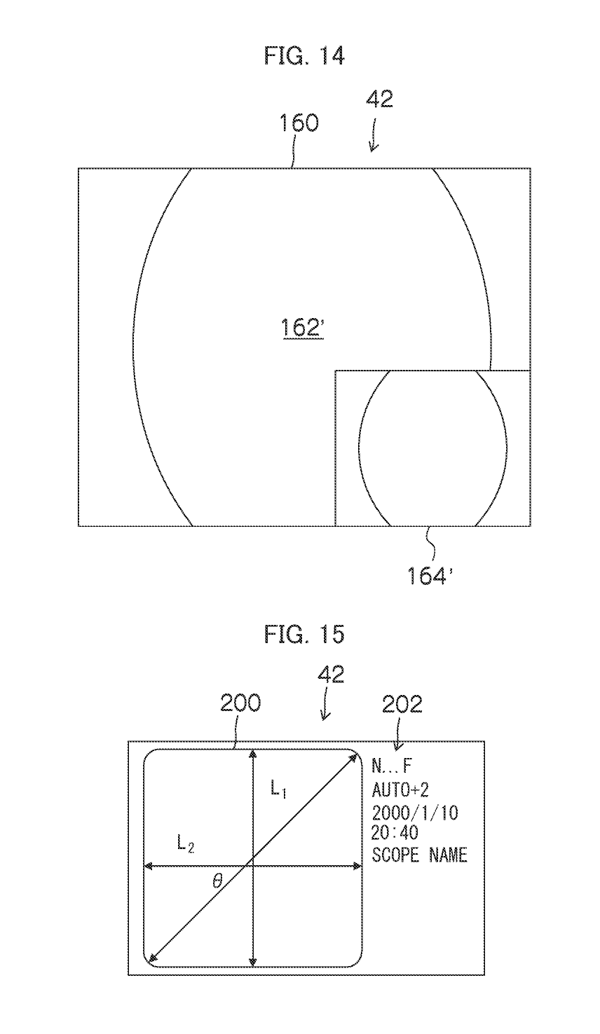

FIG. 14 illustrates a screen layout according to another variation. As illustrated in FIG. 14, a main screen region 162′ is displayed as a full screen display and a sub-screen region 164′ is arranged in one of the four corners of the screen 160 so as to be overlaid on the main screen region 162′. Note that a field mask is placed in the main screen region 162′ and the sub-screen region 164′ illustrated in FIG. 14.

Specifically, a field mask having a round shape may be placed in the main screen region 162′ or the sub-screen region 164′ may be displayed as a PiP (picture in picture) image (display). Further, the above display format and a parallel display format of the main screen region 162′ and the sub-screen region 164′ may be configured to be selectively switchable.

(Description of Field Mask)

The endoscope apparatus 1 according to the present embodiment is configured to be switchable between an isotropic mask display having a constant field of view and having the same vertical and horizontal sizes and an anisotropic mask display having the same field of view as that of the isotropic mask and having different vertical and horizontal sizes. FIG. 15 is an explanatory drawing schematically illustrating a screen 200 displaying an isotropic mask. The screen 200 illustrated in FIG. 15 has a field mask serving as the outer frame of an image to be displayed. The size L₁ of a vertical side of the field mask is the same as the size L₂ of a horizontal side thereof. An information display region 202 for displaying imaging information is arranged on the right side of the screen 200 illustrated in FIG. 15.

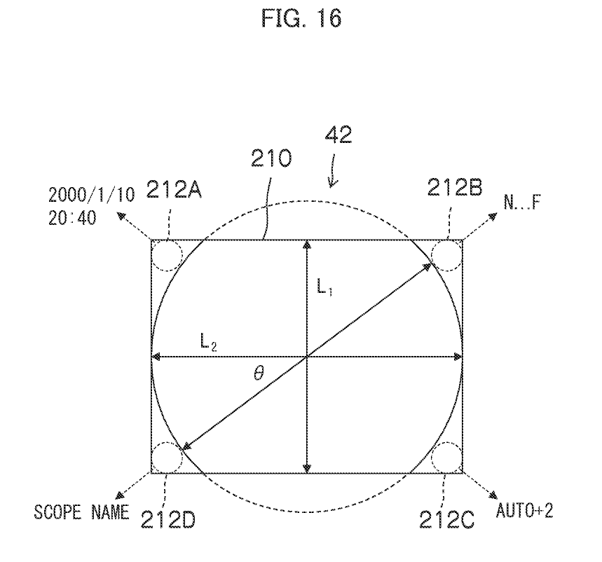

FIG. 16 is an explanatory drawing schematically illustrating a screen 210 displaying an anisotropic mask. The screen 210 illustrated in FIG. 16 has a field mask having the same field of view as that of the field mask illustrated in FIG. 15. The size L₁ of a vertical side of the field mask is smaller than the size L₂ of a horizontal side thereof. Further, information display regions 212A, 212B, 212C, and 212D for displaying imaging information are disposed on four corners of the screen 210 illustrated in FIG. 16. Various information such as imaging information is divided and displayed in the information display regions 212A, 212B, 212C, and 212D.

The description of the above embodiments has focused mainly on a medical endoscope, but the presently disclosed subject matter can be applied to a borehole observation apparatus for industrial purposes.

Hereinbefore, the endoscope apparatus according to the presently disclosed subject matter have been described in detail, but the presently disclosed subject matter is not limited to the above embodiments. It is apparent that various improvements and modifications may be made to the presently disclosed subject matter without departing from the spirit and scope of the presently disclosed subject matter.

### APPENDIX

As has become evident from the above detailed description of the embodiments of the presently disclosed subject matter, the present description includes disclosure of various technical ideas including at least the invention as follows.

(First Aspect):

An endoscope apparatus including: an illumination device emitting light to an object; an imaging device generating a color imaging signal by imaging the light reflected from the object irradiated by light emitted from the illumination device; a normal image generation device generating a normal image by performing normal image processing on the imaging signal obtained by the imaging device; a spectral image generation device generating a spectral image corresponding to a preset optical narrow band by subjecting the imaging signal obtained by the imaging device to matrix processing using matrix data of the optical narrow band; a display device displaying a main screen region and a sub-screen region having a size less than the size of the main screen region; a switching signal generation device generating a switching signal for switching a display content of the main screen region and the sub-screen region; and a display control device controlling a screen display of the display device such that the normal image is displayed in any one of the main screen region and the sub-screen region, while the spectral image is displayed in the other region, and based on the switching signal generated by the switching signal generation device, control is performed so as to selectively switch between a region for displaying the normal image and a region for displaying the spectral image.

The first aspect provides a display device having a main screen region and a sub-screen region having a size less than the size of the main screen region, wherein either one of the normal image and the spectral image is displayed in one region and an image different from the image displayed in the one region is displayed in the other region, thus allowing the operator's attention to be concentrated on one region during simultaneous observations of the normal image and the spectral image.

In a state in which a normal image is displayed in the main screen region and a spectral image is displayed in the sub-screen region, the operator can view the display of the sub-screen region to determine whether a close investigation is required or not. If required, the operator can switch the display of the main screen region from the normal image to the spectral image. Further, in a state in which a spectral image is displayed in the main screen region and a normal image is displayed in the sub-screen region, the operator can view the normal image during observation of the spectral image, thereby securing safety during observation of the spectral image having less information than the normal image.

(Second Aspect):

The endoscope apparatus according to the first aspect, wherein the display control device fixes a size and a display position of the main screen region and displays the main screen region on the display device.

According to the above aspect, the size and the position of the main screen region are fixed. Thus, the operator can easily focus his or her attention on the image displayed in the main screen region to be mainly observed.

(Third Aspect):

The endoscope apparatus according to any one of the first and second aspects, further including a sub-screen region move operation device operated to move the display position of the sub-screen region, wherein the display control device causes the display device to display the sub-screen region so as to move the display position of the sub-screen region according to the operation of the sub-screen region move operation device.

According to the above aspect, the operator can surely view both an image displayed in the main screen region and an image displayed in the sub-screen region during simultaneous observations.

(Fourth Aspect):

The endoscope apparatus according to any one of the first to third aspects, wherein the main screen region has a structure dividable into a plurality of regions, and the display control device controls the screen display of the display device so as to display a plurality of spectral images corresponding to a plurality of preset wavelength range sets in each divided region in an individual and simultaneous manner when spectral images are displayed in the main screen region.

According to the above aspect, a plurality of spectral images can be simultaneously displayed in the main screen region. Thus, the possibility of missing observation in a spectral image can be further reduced. Further, a plurality of diagnosis methods can be simultaneously applied to screening.

(Fifth Aspect):

The endoscope apparatus according to the fourth aspect, wherein one of the regions obtained by dividing the main screen region has a larger area than that of the sub-screen region.

According to the above aspect, the operator can focus on the image displayed in the main screen region more easily than the image displayed in the sub-screen region.

(Sixth Aspect):

The endoscope apparatus according to any one of the fourth and fifth aspects, wherein the display control device displays wavelength information about a spectral image displayed in each divided region.

According to the above aspect, a plurality of spectral images can be easily distinguished, thereby avoiding misconceptions of the spectral image to be focused.

(Seventh Aspect):

The endoscope apparatus according to any one of the first to sixth aspects, wherein the display device has a structure having a plurality of sub-screen regions and has a selection device selecting any one of the plurality of sub-screen regions; and the display control device controls the screen display of the display device so as to individually display a plurality of spectral images corresponding to a plurality of preset wavelength range sets determined for each of the plurality of sub-screen regions and to display, in the main screen region, a spectral image selected from the spectral images displayed in the plurality of sub-screen regions.

According to the above aspect, candidate images to be displayed in the main screen region are displayed in the sub-screen regions, thereby allowing the operator to intuitively select an appropriate spectral image.

(Eighth Aspect):

The endoscope apparatus according to the seventh aspect, wherein the display control device displays a normal image in any one of the plurality of sub-screen regions.

According to the above aspect, an image to be displayed in the main screen region can be selected from a normal image and a plurality of spectral images.

(Ninth Aspect):

The endoscope apparatus according to any one of the seventh and eighth aspects, wherein the selection device includes any one of a touch panel, a pointing device, and a scope button.

According to the above aspect, examples of the pointing device include a joystick and a mouse.

(Tenth Aspect):

The endoscope apparatus according to any one of the first to ninth aspects, wherein the main screen region and the sub-screen region have the same field of view.

According to the above aspect, the field of view is fixed. Thus, the visibility of the main screen region and sub-screen region can be maintained constant.

(Eleventh Aspect):

The endoscope apparatus according to any one of the first to tenth aspects, wherein, based on a spectral reflectance of the object, a spectral property of the illumination device, and a spectral property of the color filter of the imaging device, the spectral image generation device performs processing for estimating a reflected signal corresponding to an optical narrow band from the imaging signal corresponding to an optical wide band.

What is claimed is:

1. An endoscope apparatus comprising:
an illumination device configured to emit light to an object;
an imaging device configured to generate a color imaging signal by imaging light reflected from the object irradiated by the light emitted from the illumination device;
a normal image generation device configured to generate a normal image by performing normal image processing on the color imaging signal obtained by the imaging device;
a spectral image generation device configured to generate a spectral image corresponding to a preset optical narrow band by subjecting the color imaging signal obtained by the imaging device to matrix processing using matrix data of the preset optical narrow band;
a display device configured to display a main screen region and a sub-screen region having a size less than a size of the main screen region;
a switching signal generation device configured to generate a switching signal for switching a display content of the main screen region and the sub-screen region; and
a display control device configured to control a screen display of the display device such that the normal image is displayed in any one of the main screen region and the sub-screen region, while the spectral image is displayed in another region, and the display control device configured to control the display device so as to selectively switch between a region for displaying the normal image and a region for displaying the spectral image based on the switching signal generated by the switching signal generation device.

2. The endoscope apparatus according to claim 1, wherein
the display control device fixes a size and a display position of the main screen region and displays the main screen region on the display device.

3. The endoscope apparatus according to claim 1, further comprising:
a sub-screen region move operation device configured to be operated to move a display position of the sub-screen region, wherein
the display control device causes the display device to display the sub-screen region so as to move the display position of the sub-screen region according to the operation of the sub-screen region move operation device.

4. The endoscope apparatus according to claim 1, wherein
the main screen region is dividable into a plurality of regions, and
the display control device controls the screen display of the display device so as to display a plurality of spectral images corresponding to a plurality of preset wavelength range sets in each divided region in an individual and simultaneous manner when spectral images are displayed in the main screen region.

5. The endoscope apparatus according to claim 4, wherein
one of the regions obtained by dividing the main screen region has a larger area than that of the sub-screen region.

6. The endoscope apparatus according to claim 4, wherein
the display control device displays wavelength information about a spectral image displayed in the each divided region.

7. The endoscope apparatus according to claim 1, wherein
the display device has a plurality of sub-screen regions,
the endoscope apparatus further includes a selection device configured to select any one of the plurality of sub-screen regions, and
the display control device controls the screen display of the display device so as to individually display a plurality of spectral images corresponding to a plurality of preset wavelength range sets determined for each of the plurality of sub-screen regions and to display, in the main screen region, a spectral image selected from the spectral images displayed in the plurality of sub-screen regions.

8. The endoscope apparatus according to claim 7, wherein
the display control device displays a normal image in any one of the plurality of sub-screen regions.

9. The endoscope apparatus according to claim 7, wherein
the selection device includes any one of a touch panel, a pointing device, and a scope button.

10. The endoscope apparatus according to claim 1, wherein
the main screen region and the sub-screen region have a same field of view.

11. The endoscope apparatus according to claim 1, wherein,
based on a spectral reflectance of the object, a spectral property of the illumination device, and a spectral property of a color filter of the imaging device, the spectral image generation device performs processing for estimating a reflected signal corresponding to an optical narrow band from the imaging signal corresponding to an optical wide band.
